(19) United States
(12) Patent Application Publication
(10) Pub. No.: US 2021/0326938 A1
(43) Pub. Date: Oct. 21, 2021

(54) Mutualizing and Digitalizing for Directed Payment Systems and Methods Thereof

(71) Applicant: SERGE-ALAIN WANDJI, BROOKLYN PARK, MN (US)

(72) Inventor: SERGE-ALAIN WANDJI, BROOKLYN PARK, MN (US)

(73) Assignee: DIASPOCARE, LLC, SAINT PAUL, MN (US)

(21) Appl. No.: 16/850,784

(22) Filed: Apr. 16, 2020

(51) Int. Cl. G06Q 30/02; G06Q 20/40; G16H 50/70

(52) U.S. Cl. CPC G06Q 30/0279; G06Q 20/40145; G06F 16/2365; G06Q 20/405; G16H 50/70

(57) ABSTRACT

A method for mutualizing within an affinity group or similarly situated persons for the purposeful payment and controlling the expense of such payment for its intended purpose. The institution and relationship with partner entities are such that such funds go through a qualified merchant for use in particular areas and services as intended by the parties in the transaction. The method herein addresses the challenge of creating the buying power to negotiate and manage the delivery of services including healthcare and more.

### PRIOR APPLICATION

This application claims the benefit of U.S. Provisional Application No. 62/4834,949, entitled “Mutualizing and Digitalizing for Directed Payment Systems and Methods Thereof” filed Apr. 16, 2019 which is hereby incorporated by reference.

### BACKGROUND

In developing countries, cost of health care, education and other basic necessities are major causes of concern, especially in situations where the divide between the rich and the poor is magnified. Health care cost, being one of the most expensive, yet necessary expenditures suffer through these challenges for the low to middle income population. It is not unusual to note that a lot of people die waiting for simple treatment, simple medical issues become exacerbated and expensive as treatments and intervention are delayed or unmet. As an example, a person suffering from diabetes may have enormous complications and incur incredible expenses if the illness is not managed. The attendant high cost of intervention and the likely permanent damages (including loss of limbs) would be avoided or mitigated if the patient suffering from such chronic illness did not have to suffer through lack of economic capacity to treat or manage the illness.

Patients in developing or low income communities often rely on medical missions or non-government organizations to bridge the gap between lack of funds and availability of medical care. In most instances, these medical missions are like fire fighters trying to put out a fire with little or no capacity to prevent or manage the situations that gave rise to the fire, in the first instance. The medical missions or other well-meaning medical providers are usually late for the less expensive work that needs to be done.

Medical professionals are also leery of investing their limited resources into treating low income patients, no matter their illness, because these patients lack the capacity to pay for their necessary treatment and the medical professionals have to be paid for their work. As in most developing countries, the government usually steps in to provide medical care, and the world is awash with stories of their inefficiencies and unneeded corruption that the masses of people needing care for chronic illnesses are enough to overwhelm most medical facilities and their providers in low to middle income communities.

From the above, it is notable that the capacity to pay, advocate for a solution to these dire needs can do a world of good for the patients who may have some small amounts of money but either cannot save enough for the treatment, or are lacking the resources to pool together a system for the delivery of these needed services. Such a system allowing for the collective to further the provision of these types of care and access is disclosed herein.

### SUMMARY

In one embodiment, the present invention provides a novel method for mutualizing the financial contributions of an affinity group, digitalizing such contributions and rotating the beneficiaries of such rotation until all members of the affinity group has their turn, wherein such rotation for the beneficiaries provide the resources for the purposeful payment to serve the cost of treatment.

### BRIEF DESCRIPTION OF THE INVENTION

The mutualizing process according to the present invention showing an affinity group collective for the purpose of generating contributions for the purpose of addressing the needs of the affinity group members. The affinity group according to the present invention may be a select set of participants with ailments in common. As an example, diabetes mellitus sufferers may get together as an affinity group of diabetes patients in a particularized group. Registration and identification of members in this or any group may undergo any and all processes of registration that will provide the needed security for the members. As an example, each member of an affinity group according to the present invention may provide his or her biometric information for the added security of ascertaining that at all times, only the right user shall have access to the resources associated with the affinity group and that resource. Any and all possible biometric identification processes that are suitable to identify the appropriate owner of the contributions may be incorporated into the database to keep and accurately maintain the needed information. Biometric information herein referred to include but are not limited to: eye scan, fingerprint, voice, face identification, to name a few.

The affinity group, managing a common interest, may then apportion a certain amount of money that needs to be available for medical, pharmaceutical or other related kind of payment. Such amount of money may be needful for insurance costs, actual cost or negotiated cost of treatment or medical for the period, which may be up to one year or such selected period of coverage. Upon determining an amount that is either for the full or partial cost of medical or related care, members of the affinity group may preferably determine how much each member of the affinity group would need to contribute as a total sum, monthly or just periodically. As an example, a twelve-member affinity group of diabetes patients may contribute one-twelfth of the total amount needed to pay for a yearly coverage of their sickness; each member paying in their contribution to the monthly beneficiaries' account.

Pooling the funds provides a base for negotiated medical costs and answers the challenges for medical professionals needing to be paid for their services and may be enough to use as a negotiating tool to reduce the cost of healthcare and promote health access given the assurances of payment after service is provided. This aggregation of funds provides leverage to meet the needs of the providers and beneficiaries, thus creating a much needed opportunity to reduce by economic processes, the overall uncertainties in the health care provision system, thereby impacting cost of care. The pooling and management of the contributions according to the present invention act like a pledge, credit or commitment to pay for the stakeholders in the health care system.

A schematic of the steps according to an embodiment of the present invention may present a patient or customer who is accredited a member of an affinity group and may have gone a hospital or clinic to address a medical need. At a clinic or hospital, the patient is preferably issued a prescription for medicament by, preferably a medical practitioner. Reasonably, the patient or customer may have a previously approved prescription in the system of the present invention. The next sequence in the process is preferably a visit to a pharmacy or pharmacist with the issued prescription. At the pharmacy, a prescription is created in the system of the present invention and notification is made to the buyer, if known. The pharmacist according to the present invention should have been registered with the system of the present invention prior to the transaction of directed payment system. The pharmacy is preferably the location for information about the transaction of completing the delivery of medicament to the patient, maintaining online versions of the prescriptions, creation of an electronic transaction, and maintaining an accounting of the transactions including amount escrowed or utilized.

An affinity management process or operator may be contacted by the patient or pharmacy or agent of same according to the present invention. It is reasonable to indicate that the operator of the system may complete a transaction according to the present invention without the formal registration process. The registration process is preferred as it allows for easy and smooth processing as it should provide the accommodation to authenticate the validity of the member of the affinity group. It is also reasonable to build on the system as it provides a record of medical care provided and springs the process of creating a medical record system for the patient member of the affinity group. Such record may then be available for reference or recall at any other location of choice as needed for the improvement of health care and health care records.

Upon the verification of the membership or affiliation with the affinity group, the transaction is transmitted to the pharmacy wherein the medicine or product paid for is available for delivery to patient or client member. A confirmation of the transaction and completion of the process of the present invention is included in the succeeding step wherein the buyer is notified. Where confirmation proceeding cannot be electronically completed, other processes may be incorporated to provide such assurance as needed.

The process begins with the patient or client, pharmacy and affinity group operator, having been enrolled in the system of the present invention. In the system of the present invention, patient or client submits a prescription at the pharmacy wherein all aspects necessary for the process according to the present invention are entered or documented according the requirements of the network or system. If said system is previously not populated with all required information, the buyer or affinity group operator may be referred for review.

The provided information is then preferably verified for completeness and readiness for the transaction and completion of same. When all determined aspects of the process are verified as complete, the parties to the transaction at the origination and delivery points are confirmed and medicine or medical care delivered. Where the delivery of medicine or care is not practical for any reason, the notification for the system is reverted to note the reason(s) for non-delivery or non-performance and document verification. The cycle may continue until a successful outcome is achieved. Upon delivery of the medicament or care, the system updates are attained.

The present invention discloses a system for mutualizing contributions within an affinity group of similarly situated persons for providing directed payment and control for use of such purposeful payment by the beneficiary and agents associated therewith. In the instant disclosure, the term “directed” or “purposeful” payment is used interchangeably to imply payment obtained or facilitated for the distinct purpose intended, in essence, assuring the ‘last mile’ in the delivery of health care or other needful services or items as demanded by the affinity group. Everyday payment for services is premised on the payer and the actual beneficiary receiving the funds. In most instances, the use or application of such funds is of no consequence to the service provider as long as the transaction is completed according to the terms of the service. The present invention serves the need for an affinity group and the purposeful or directed payment and use of such funds in a manner anticipated by the parties.

In an embodiment of the present invention, a need arises with a purpose; said purpose being, in the primary, getting access to services for a member of an affinity group. Such services may range from paying for medicaments, purchasing groceries, school expenses, building a house, other services, and the like. At the establishment of such need, and the trigger of the system of the present invention, a process for getting the now purposeful funds pledged or committed by an affinity group to serve the need of members of the affinity group is set in motion. The present invention provides a system to identify the payer, confirm the need, express the funds, ensure such funds are applied towards the need, and confirm such application to the payer with the confidence that the execution is preferably transacted in a manner prescribed.

Affinity groups may be located in the location of need or anywhere else, either created by the patients or in some instances by the funders of the system who may be anywhere in the world. It is imaginable that a group of sponsors in the Diaspora may team up to create an affinity group to fund or commit for the funding of treatments for needy persons anywhere the system of the present invention is applicable. Non-government organizations may support this process and in practical terms become the affinity group that caters to needy beneficiaries. These processes may practically be harnessed for the leverage needed to impact the cost of health care or similarly situated needs in the marketplace according to the present invention. Hereinafter, the affinity group and Diaspora group may be addressed interchangeably.

In the first instance, a payer may establish, sign up or link into the system of the present invention in any manner provided by this invention. The present invention provides a means for preferably establishing a link or connecting into a proprietary system and identifying a potential user who may need to register or otherwise identify as a user. It is reasonable to expect that an enrollee or registrant may provide some personally identifiable information prior to use either in the registration process or for later use to ensure and assure the integrity of the service. The system whereby a user links into is preferably accepted as a network of providers of such expected service, whether in the location of the payer or in the locale of the beneficiary. Expressed otherwise, the user is preferably using a network of providers, either by physical structure or by partnerships to effect the service such as provided in the system according to the present invention.

Upon readiness of a user to exploit the benefits of the present invention, a need is preferably identified. Such need may be, as an example, the payment for medicament in the country of the beneficiary. In this instance, a pharmacy or service provider in the locale of the beneficiary, who apriori, shall have registered and was approved as a provider in the network, is alerted, either by the system or beneficiary of the system to the need to provide the expected medicament as authorized by the parties in the process. The use of the system or effecting of the process may have been occasioned by the establishment of a medical or pharmaceutical need in the appropriate instance. Having all the expressed prerequisites in place to establish the authenticity, availability and capacity to serve the need, a system is preferably triggered to effect the authority to provide such service of funding to meet the need.

Any establishment participating in the purposeful transfer of funds according to the present invention, shall, of necessity, possess the resources to effect, on demand, the outcomes expected to meet the need of the beneficiary at the opportune time and report such completion within the network and system of the present invention. Funding for the service according to the present invention may be provided at the onset, by the payer, either via a deposit, credit or debit card, or by any other prescribed means established by the purveyors of the system at the time of use. It is reasonable to expect a relationship within the network of funded or pre-funded providers who can, with minimal notice, provide in the locale of the beneficiary, the needed funding or the goods as prescribed. Further, it is reasonable to have service providers with accounts that are suitable for replenishing at the locale of the beneficiary. The expression of the funding to meet the purposeful payment according to the present invention may be automatic or occasioned after the manual verifications necessary to ensure authenticity. Some options for transfer or payment for medicament is disclosed in the drawings or flow charts submitted herein.

The assurance of the purposeful or directed payment of the present invention preferably reduces the sometimes negative impact of middlemen or facilitators in the process of delivering the intended funds to the intended beneficiary and for the intended purpose. The funding according to the present invention remains in the possession of providers of services as against middlemen who may be tempted to act in their self-interest instead of the primary interest of the beneficiary. The provider of services in the locale of the beneficiary may, in the instance of medicament, dispense the medicaments or drugs that are prescribed and paid for, instead of the extras added. It is an object of the present invention to verify and ascertain that the purpose of each transaction is met at each instance.

A benefit of the present invention is verifiable service, and provided in the at least a process usable to verify the deliverables expected at each decision point in the purposeful payment. The system of the present invention provides monitoring mechanisms, whether electronically or otherwise, for the beneficiary and payer to document success in a reportable manner for use in verification and the attendant data analyses. Such verification builds success and confidence and may provide a means to predict successful outcomes from specific tasks.

Use of the system of the present invention is not predicated on having a bank account or other accounts which may or may not be a barrier to a user. Most affinity group members may be able to take advantage of the process simply by being a member of such group and the limitation of having a bank account is thus not a barrier to entry.

Another benefit of the present invention is the provision of a system that may become the precursor to medical record keeping, consumer price indices for goods and services, more robust data collection and polling in the location of the beneficiary, especially where such medical records where previously unavailable.

A pharmacist or service provider at the beneficiary's locale may obtain information as needed to satisfy the terms and requirements of the network according to the present invention. When the service is provided at the locale of the beneficiary according established expectations, the system is concurrently updated to reflect such transactions, and the attendant information pertaining to same.

On the Diaspora end, the payer or affinity group provides the funding for the particular and intended service prior to the delivery of said service, unless previously approved otherwise. Payment for the service or product may be effected using current electronic payment processes now known or later determined. Upon establishment in the system of payment by the sponsor or payer, the beneficiary may preferably obtain the product or service at the delivery point almost instantaneously. The system of the present invention is preferably suitable to notify and update all pertinent parties without the challenge of effecting a fund wire transfer at the instance of service as is obtainable with banks and other financial service organizations. According to the present invention, the payer funds the system and the beneficiary obtains the service or product without the challenge of going to financial institutions to obtain the monies for such desired transactions.

Accordingly, the present invention provides a system for a marketplace of service providers, who given prior systematic processes, uses a pre-arranged process to fund a provider and manage the collection of information and delivery of service or product to the beneficiaries. The system of the present invention is preferably useful for the direct or purposeful payment for medicaments, building material, education, groceries, clothing, and others. The description provided herein indicates and illustrates the flow and design structure of an aspect of the present invention for use in the delivering of purposeful payments for medical services and medicaments. The system includes the value and services provided by pharmaceutical companies and groups offering related services for the medical industry.

Another benefit of the present invention is that it uses the increasing mobile applications, technology and the internet to drive down the transaction cost for delivering needed services and providing a greater share of their business. Specifically, pharmacies in developing communities would be very interested in increasing their customer base and this invention enables them to increase their revenues from a segment of the market that would otherwise not have the means to afford their medicines.

The present invention provides a means to soften the impact of high cost of medicine to the beneficiaries and prevents them from falling down into extreme poverty. In other instances, the cost of education can be paid or subsidized by the donors for the direct use of the beneficiaries. As the global market place becomes smaller, money flows from affinity groups to pay merchants in developing and low-income communities for goods and services for consumers in developing communities increases partnerships and likely upgrades the standard of living in the recipient communities.

According to the present invention, a system is provided wherein information about the beneficiary may be obtained. As an example, the present invention provides a convenient access to obtain medical records of the beneficiaries as they process their need for medicine from the service providers. Such medical records may include ailments, medication provided to serve that need, the prevalence of certain type of ailments, etc.

A benefit of the present invention is that the service providers impacted by the teachings of the present invention would be interested because the influx of new revenue helps boost their economy. These and other applications are available from the teachings of the present invention.

The mutualizing within an affinity group aggregates the funds to enable bargaining power to ensure both quality and lower cost of medicines and healthcare services. Utilizing the mutualizing process provides an approach to achieve quality at a low cost. As an example, a mutualizing for diabetes care may operably be focused to secure a specific medicine brand (sometimes referred to as a formulary) directly from a trusted distributor or manufacturer at an efficient or affordable cost. Such medicine may then be picked up at closest pharmacy or delivered directly to a patient.

In another example, a mutualizing case may be use aggregated funds to purchase a number of “doctor-hours” at an affordable rate A concierge doctor may be made available to come to a location for the provision of medical care to all mutualized participants or in the alternative, an appointment is secured for such participants to visit the medical practitioner at a location of choice.

In yet another example, this mutualized process may also be used to purchase or attain at least one or more subsequent opinions on a particular medical concern from medical practitioners in the Diaspora or where such preferred opinion is sought at an affordable cost. (“Second-Opinion Doctor-Hours”).

Inventory management may be included as part of the package or provision of the current invention and may be usable pre and post-delivery of medicine or drugs. Other avenues may be conducted using a pharmacy inventory management system.

Double authentication system wherein the authentication of the beneficiary is confirmed (biometrically for example) and said identity matched with his or her electronic health record before payment is completed.

A mutualizing app may also be used to help Diaspora members or associations digitize their current mutualizing system for optimum efficiency, convenience and also security—eliminating the need to transport large sums of currencies or local denominations to the mutualizing meeting or post meeting and saving the risk of transporting same to deposit at a bank or financial institution.

In another instance, the mutualizing according to the present invention may aid Diaspora associations aggregate the funds to purchase health insurance at a lower rate, or to meet their deductibles, or purchase supplemental health insurance, or to pay for funeral expenses of loved ones who pass away at the beneficiary's location.

Having thus described the preferred embodiments of the present invention, those of skill in the art will readily appreciate that the teachings found herein may be applied to yet other embodiments.

What is claimed:

1. A method of mutualizing a payment system, comprising:
selecting an affinity group, wherein said group further comprises at least two participants having at least a common reason to be part of such affinity group;
determining at least a rule or frequency for such affinity group payment rotation;
establishing a contribution procedure for such payments and contributing to same;
digitizing said affinity group payment to meet said affinity group medication or treatment needs;
wherein the contributions and payment process are mutualized for the purposeful payment to serve a cost of treatment and medication.

2. The method of claim 1, wherein membership or registration in the affinity group is verified by biometric information.

3. The method of claim 1, wherein the purpose of the affinity group is limited to medical treatment and related transactions.

4. The method of claim 1, wherein the affinity group members are registered with a directed or purposeful payment process for the dispensation of medical treatment or related transaction.

5. The method of claim 1, wherein the payment system is directed to assuring the last mile in the delivery of health care.

6. The method of claim 1, wherein the directed or purposeful payment system is applied to other needs such as education or home building.
